(19) United States
(12) Patent Application Publication
(10) Pub. No.: US 2009/0131485 A1
(43) Pub. Date: May 21, 2009

(54) Deuterated pirfenidone

(75) Inventors: Julie F. Liu, Lexington, MA (US); Yong Dong, Wayland, MA (US)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: CoNCERT Pharmaceuticals, Inc., Lexington, MA (US)

(21) Appl. No.: 12/283,290

(22) Filed: Sep. 10, 2008

Related U.S. Application Data
(60) Provisional application No. 61/019,481, filed on Jan. 7, 2008.
(60) Provisional application No. 60/971,083, filed on Sep. 10, 2007.

(51) Int. Cl. A61K 31/4402; C07D 213/64; C12N 5/02

(52) U.S. Cl. 514/345; 546/290; 435/375

(57) ABSTRACT

This invention relates to novel substituted pyridinones, their derivatives, pharmaceutically acceptable salts, solvates, and hydrates thereof. This invention also provides compositions comprising a compound of this invention and the use of such compositions in methods of treating diseases and conditions that are beneficially treated by administering a TNF (tumor necrosis factor)-alpha production inhibitor/TGF (transforming growth factor)-beta inhibitor.

### RELATED APPLICATIONS

This application claims the benefit of U.S. Provisional Application No. 61/019,481, filed on Jan. 7, 2008 and U.S. Provisional Application No. 60/971,083, filed on Sep. 10, 2007.

The entire teaching of the above applications are incorporated herein by reference.

### BACKGROUND OF THE INVENTION

Pirfenidone, also known as 5-methyl-1-phenylpyridin-2(1H)-one, is thought to inhibit collagen synthesis, down-regulate multiple cytokine production, and block fibroblast proliferation and stimulation in response to cytokines.

Pirfenidone is currently pre-registered for idiopathic pulmonary fibrosis (IPF) in Japan, and is in clinical trials for IPF in Europe and the US. It is also being investigated for neurofibromatosis, Hermansky-Pudlak syndrome, diabetic nephropathy, renal failure, hypertrophic cardiomyopathy (HCM), glomerulosclerosis (FSGS), radiation-induced fibrosis, multiple sclerosis, and uterine leiomyomas (fibroids).

Adverse events experienced by patients dosed with pirfenidone include, but are not limited to, nausea, gastrointestinal disturbances, fatigue, headache, photosensitive skin rash, and moderate photosensitivity (Raghu, G et al., Am J Resp Crit. Care Med, 1999, 159(4):1061. Thus, despite the beneficial activities of pirfenidone, there is a continuing need for new compounds to treat the aforementioned diseases and conditions.

### SUMMARY OF THE INVENTION

This invention relates to novel substituted pyridinones, their derivatives, pharmaceutically acceptable salts, solvates, and hydrates thereof. This invention also provides compositions comprising a compound of this invention and the use of such compositions in methods of treating diseases and conditions that are beneficially treated by administering a TNF (tumor necrosis factor)-alpha production inhibitor and/or TGF (transforming growth factor)-beta inhibitor.

### BRIEF DESCRIPTION OF THE DRAWINGS

FIG. 1 depicts the pharmacokinetics of a compound of this invention as compared to pirfenidone following intravenous administration in rats.

FIG. 2 depicts the pharmacokinetics of a compound of this invention as compared to pirfenidone following oral administration in rats.

FIG. 3 depicts the pharmacokinetics of compounds of this invention as compared to pirfenidone following intravenous administration in chimps.

FIG. 4 depicts the pharmacokinetics of compounds of this invention as compared to pirfenidone following oral administration in chimps.

FIG. 5 depicts the pharmacokinetics of a compound of this invention as compared to pirfenidone following intravenous administration in rats.

FIG. 6 depicts the pharmacokinetics of a compound of this invention as compared to pirfenidone following intravenous administration in rats.

FIG. 7 depicts the pharmacokinetics of a compound of this invention as compared to pirfenidone following oral administration in rats.

FIG. 8 depicts the pharmacokinetics of a compound of this invention as compared to pirfenidone following oral administration in rats.

### DETAILED DESCRIPTION OF THE INVENTION

The terms “ameliorate” and “treat” are used interchangeably and include both therapeutic and prophylactic treatment. Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

It will be recognized that some variation of natural isotopic abundance occurs in a synthesized compound depending upon the origin of chemical materials used in the synthesis. Thus, a preparation of pirfenidone will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Ganes L Z et al., Comp Biochem Physiol Mol Integr Physiol 1998, 119:725. In a compound of this invention, when a particular position is designated as having deuterium, it is understood that the abundance of deuterium at that position is substantially greater than the natural abundance of deuterium, which is 0.015%. A position designated as having deuterium typically has a minimum isotopic enrichment factor of at least 3000 (45% deuterium incorporation).

Unless otherwise stated, when a position is designated specifically as “H” or “hydrogen”, the position is understood to have hydrogen at its natural abundance isotopic composition.

The term “isotopic enrichment factor” as used herein means the ratio between the isotopic abundance of D at a specified position in a compound of this invention and the naturally occurring abundance of that isotope. The natural abundance of deuterium is 0.015%.

In one embodiment, each position designated specifically as “D” or “deuterium” has an isotopic enrichment factor of at least 3340 (at least 50.1% incorporation of deuterium at that position). Thus, the resulting compound has an isotopic enrichment factor of at least 3340.

In other embodiments, a compound of this invention has an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

The term “isotopologue” refers to a species that differs from a specific compound of this invention only in the isotopic composition thereof. Isotopologues can differ in the level of isotopic enrichment at one or more positions and/or in the positions(s) of isotopic enrichment.

The term “compound,” when referring to the compounds of the invention, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain minor amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound. However, as set forth above, typically the relative amount of such isotopologues in toto will be less than 55% of the amount of the compound (i.e., the particular structure depicted will represent at least 45% of the isotopologues that make up the compound). In other embodiments, the relative amount of such isotopologues in toto will be less than 49.9%, less than 47.5%, less than 40%, less than 32.5%, less than 25%, less than 17.5%, less than 10%, less than 5%, less than 3%, less than 1%, or less than 0.5% of the compound.

The invention also includes solvates and hydrates of the present invention.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

As used herein, the term “hydrate” means a compound which further includes a stoichiometric or non-stoichiometric amount of water bound by non-covalent intermolecular forces. Examples of specific hydrates include those hydrates that are known to form with respect to the non-deuterated versions of the present compounds.

As used herein, the term “solvate” means a compound which further includes a stoichiometric or non-stoichiometric amount of solvent such as water, acetone, ethanol, methanol, dichloromethane, 2-propanol, or the like, bound by non-covalent intermolecular forces. Examples of specific solvates include those hydrates that are known to form with respect to the non-deuterated versions of the present compounds.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“D” refers to deuterium. “Stereoisomer” refers to both enantiomers and diastereomers. “Tert”, “^{t}”, and “t-” each refer to tertiary.

Throughout this specification, a variable may be referred to generally (e.g., “each R”) or may be referred to specifically (e.g., R¹, R², R³, etc.). Unless otherwise indicated, when a variable is referred to generally, it is meant to include all specific embodiments of that particular variable.

### Therapeutic Compounds

The present invention provides a compound of Formula I:

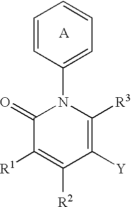

or a pharmaceutically acceptable salt, hydrate or solvate thereof, wherein:

ring A is a phenyl ring having zero to five deuterium;

each of R¹, R² and R³ is independently selected from H or D; and

Y is selected from CH₂D, CHD₂, or CD₃, and when at least one of R¹, R², or R³ is D, or when ring A has at least one deuterium, Y is additionally selected from CH₃.

In one embodiment, the invention provides a compound wherein Y is selected from CH₂D, CHD₂, or CD₃.

One embodiment provides a compound of Formula I wherein ring A has zero or five deuterium.

Another embodiment provides a compound of Formula I wherein Y is CH₃ or CD₃.

Another embodiment provides a compound of Formula I wherein Y is CD₃ and ring A has zero or five deuterium.

Another embodiment provides a compound of Formula I wherein Y is CD₃ and ring A has zero deuterium.

In yet another embodiment, the compound is selected from any one of the following:

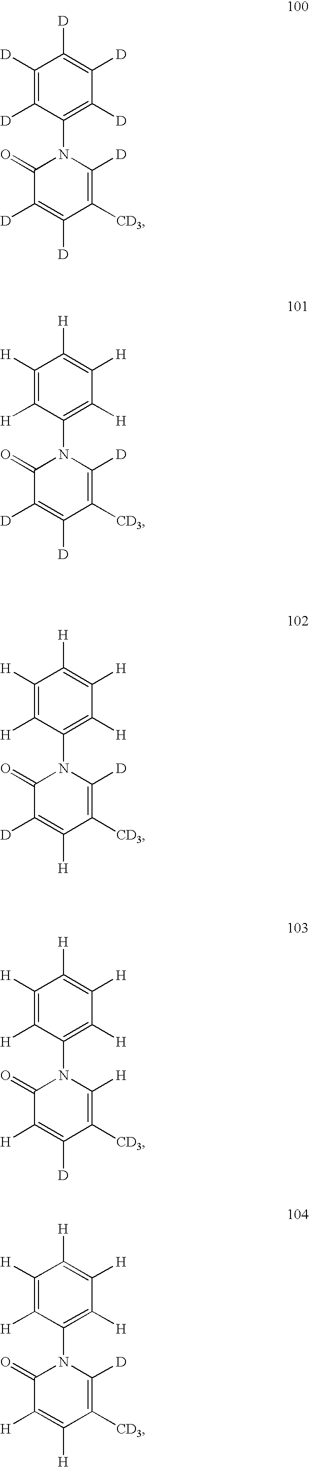

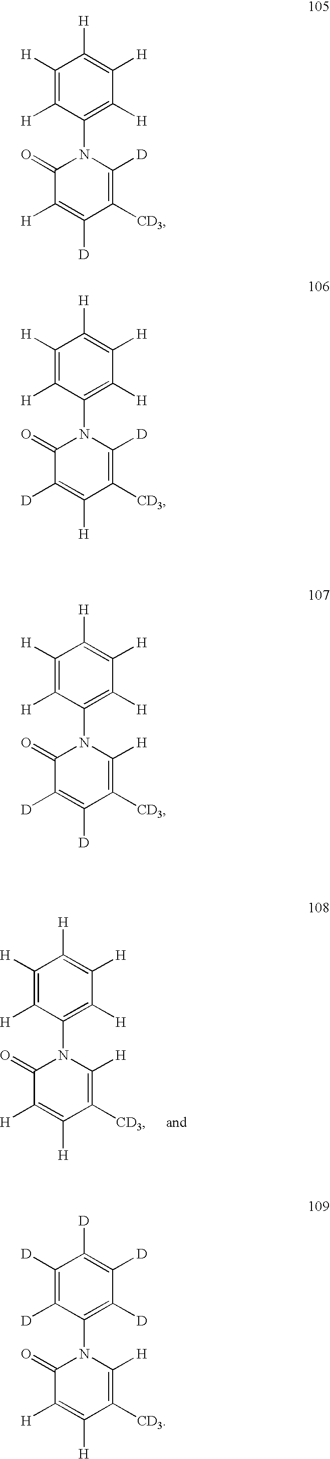

In another set of embodiments, any atom not designated as deuterium in any of the embodiments set forth above is present at its natural isotopic abundance.

The synthesis of compounds of Formula I can be readily achieved by synthetic chemists of ordinary skill. Relevant procedures and intermediates are disclosed, for instance in Castaner, J et al., Drugs Fut 1977, 2(6):396; Chinese Patent Application Nos CN 1817862, and CN 1386737; and PCT Patent publication No. WO 2003014087.

Such methods can be carried out utilizing corresponding deuterated and optionally, other isotope-containing reagents and/or intermediates to synthesize the compounds delineated herein, or invoking standard synthetic protocols known in the art for introducing isotopic atoms to a chemical structure. Certain intermediates can be used with or without purification (e.g., filtration, distillation, sublimation, crystallization, trituration, solid phase extraction, and chromatography).

### Exemplary Synthesis

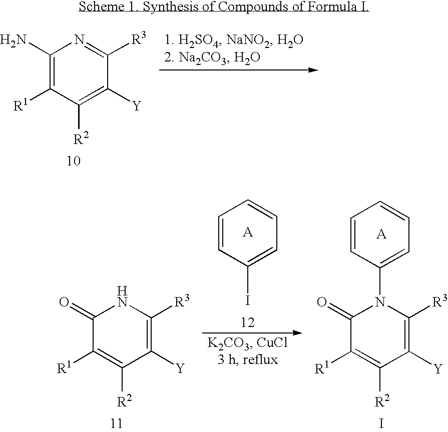

A convenient general method for synthesizing compounds of Formula I is depicted in Scheme 1. An appropriately deuterated aminopyridine 10 is oxidized to the corresponding pyridinone 11. The pyridinone 11 is then combined with an appropriately deuterated iodobenzene 12 to produce a compound of Formula I.

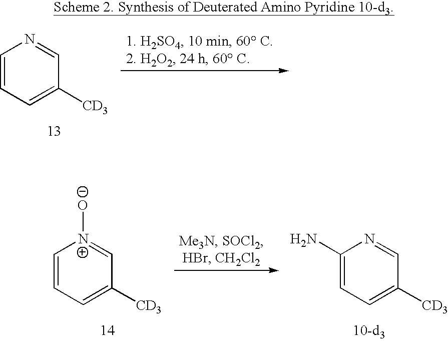

Scheme 2 shows a route for making a deuterated aminopyridine 10-d₃ useful in Scheme 1, wherein R¹, R², and R³ are H; and Y is CD₃. The scheme follows the general method set forth in Japanese Patent publication JP2005255560. Commercially-available 3-(methyl-d₃)-pyridine (13) is oxidized to the corresponding N-oxide 14, which is then be converted to aminopyridine 10-d₃ via the general method disclosed in German Patent publication DE4232175. Alternatively, 3-(methyl-d₃)-pyridine (13) may be treated with n-BuNH₂, followed by HBr to produce deuterated amino pyridine 10-d₃ following the method disclosed in U.S. Pat. No. 4,405,790.

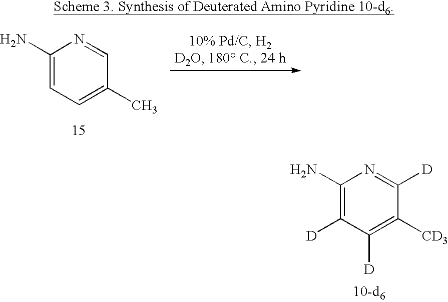

Scheme 3 shows a route for making deuterated aminopyridine 10-d₆ that is useful in Scheme 1, wherein R¹, R², and R³ are D; and Y is CD₃. The phenyl hydrogens in commercially available 2-amino-5-methylpyridine (15) are catalytically exchanged for deuteriums using activated Pd/C and D₂O to produce 10-d₆ See H Esaki, et al, Tetrahedron 2006, 62:10954-10961.

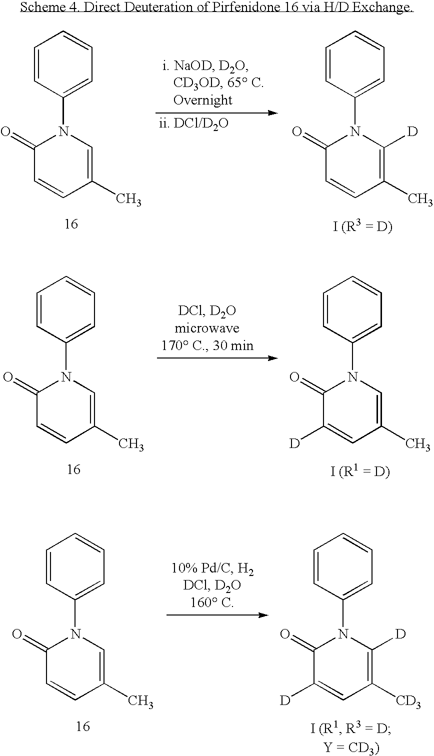

Scheme 4 shows various reactions for the direct deuteration of pirfenidone (16) via H/D exchange under different conditions to provide different compounds of Formula I. Treatment of 16 with NaOD in D₂O/CD₃OD produces a compound of Formula I, wherein ring A contains no deuterium; R¹, and R² are H; Y is CH₃; and R³ is D. Treatment of 16 with DCl in D₂O in a microwave reactor at 170° C. produces a compound of Formula I wherein ring A contains no deuterium; R¹ is D, R² and R³ are H, and Y is CH₃. Treatment of 16 with 10% palladium on carbon in the presence of H₂ and D₂O following the general methods of H Esaki, et al, Tetrahedron 2006, 62:10954-10961 produces a compound of Formula I wherein ring A contains no deuterium; R¹ and R³ are D; R² is H; and Y is CD₃.

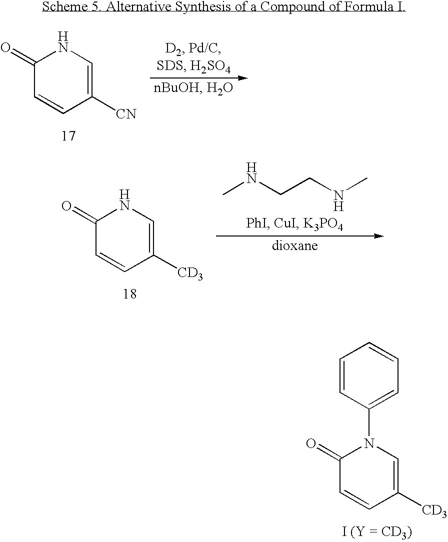

Scheme 5 shows an alternate route for producing a compound of Formula I, wherein Y is CD₃ and each of R¹, R² and R³ is hydrogen. Commercially available 6-oxo-1,6-dihydropyridine-3-carbonitrile (17) and sodium dodecyl sulfate (“SDS”) and sulfuric acid are dissolved in n-butanol/water and hydrogenated with deuterium gas over palladium on carbon to produce 5-(methyl-d₃)-pyridin-2(1H)-one 18. The use of deuterated solvents and reagents, such as D₂SO₄, nBuOD and D₂O, provides 18 in which the isotopic abundance is improved. The pyridinone 18 is then treated with iodobenzene, copper (I) iodide, N,N′-dimethylethylenediamine and K₃PO₄ to produce a compound of Formula I, wherein Y is CD₃; and each of R¹, R² and R³ is hydrogen. In Scheme 5, PhI may also represent a deuterated version of iodobenzene.

Yet another way of producing 5-(methyl-d₃)-pyridin-2(1H)-one 18, and a compound of Formula I wherein Y is CD₃, is set forth in Example 6.

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹, R², R³, etc.) or not. The suitability of a chemical group in a compound structure for use in the synthesis of another compound is within the knowledge of one of ordinary skill in the art.

Additional methods of synthesizing compounds of Formula I and their synthetic precursors, including those within routes not explicitly shown in schemes herein, are within the means of chemists of ordinary skill in the art. Methods for optimizing reaction conditions and, if necessary, minimizing competing by-products, are known in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3rd Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof.

Combinations of substituents and variables envisioned by this invention are only those that result in the formation of stable compounds.

### Compositions

The invention also provides pyrogen-free compositions comprising an effective amount of a compound of Formula I (e.g., including any of the formulae herein), or a pharmaceutically acceptable salt, solvate, or hydrate of said compound; and an acceptable carrier. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J Hauss, ed. Informa Healthcare, 2007; and “Role of Lipid Excipients in Modifying Oral and Parenteral Drug Delivery: Basic Principles and Biological Examples,” Kishor M Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the patient, such organ or tissue may be bathed in a medium containing a composition of this invention, a composition of this invention may be painted onto the organ, or a composition of this invention may be applied in any other convenient way.

In another embodiment, a composition of this invention further comprises a second therapeutic agent. The second therapeutic agent may be selected from any compound or therapeutic agent known to have or that demonstrates advantageous properties when administered with a compound having the same mechanism of action as pirfenidone. Such agents include those indicated as being useful in combination with pirfenidone, including but not limited to, those described in WO 2004019863, WO 2004105684, WO 2005013917, WO 2005038056, and WO 2005110478.

Preferably, the second therapeutic agent is useful in the treatment of a patient suffering from or susceptible to a disease or condition selected from idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; glomerulosclerosis (FSGS); radiation-induced fibrosis; multiple sclerosis, including secondary progressive multiple sclerosis; uterine leiomyomas (fibroids); alcoholic liver disease including hepatic steatosis, hepatic fibrosis and hepatic cirrhosis; keloid scarring; hepatitis C virus (HCV) infection; proliferative disorders, including angiogenesis-mediated disorders, cancer (including glioma, glioblastoma, breast cancer, colon cancer, melanoma and pancreatic cancer) and fibrotic disorders; interstitial lung diseases; atrial fibrillation (AF); organ transplant rejection; and scleroderma and related fibrotic conditions of the skin.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the pharmaceutical compositions of the invention, the compound of the present invention is present in an effective amount. As used herein, the term “effective amount” refers to an amount which, when administered in a proper dosing regimen, is sufficient to treat (therapeutically or prophylactically) the target disorder. For example, to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother Rep 50: 219. Body surface area may be approximately determined from height and weight of the patient. See, e.g., Scientific Tables, Geigy Pharmaceuticals, Ardsley, N.Y., 1970, 537.

In one embodiment, an effective amount of a compound of this invention can range from about 2 to about 8000 mg per treatment. In more specific embodiments the range is from about 20 to 4000 mg or from 40 to 1600 mg or most specifically from about 200 to 800 mg per treatment. Treatment typically is administered one to three times daily. In another embodiment, an effective amount of a compound of this invention is between about 800 to 2400 mg/day.

Effective doses will also vary, as recognized by those skilled in the art, depending on the diseases treated, the severity of the disease, the route of administration, the sex, age and general health condition of the patient, excipient usage, the possibility of co-usage with other therapeutic treatments such as use of other agents and the judgment of the treating physician. For example, guidance for selecting an effective dose can be determined by reference to the prescribing information for pirfenidone.

For pharmaceutical compositions that comprise a second therapeutic agent, an effective amount of the second therapeutic agent is between about 20% and 100% of the dosage normally utilized in a monotherapy regime using just that agent. Preferably, an effective amount is between about 70% and 100% of the normal monotherapeutic dose. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective dosage of the second therapeutic agent and/or the compound of this invention to be reduced from that required in a monotherapy. This has the advantage of minimizing toxic side effects of either the second therapeutic agent or a compound of this invention, synergistic improvements in efficacy, improved ease of administration or use and/or reduced overall expense of compound preparation or formulation.

In yet another embodiment the invention provides a pharmaceutical composition comprising a compound of Formula I and a pharmaceutically acceptable carrier, the administration of which to a test subject results in a serum terminal elimination half-life of the compound that is greater than the serum terminal elimination half-life of pirfenidone when pirfenidone is administered to an equivalent test subject in a molar equivalent pharmaceutical composition of pirfenidone under the same dosing conditions as the compound of Formula I.

In other embodiments, the serum terminal elimination half-life of a compound of Formula I is at least 110%, 120%, 130%, 140% or more of the serum terminal elimination half-life of pirfenidone produced by administration of a molar equivalent pirfenidone composition under the same dosing conditions to an equivalent test subject. In a more specific embodiment, the test subject is administered a single dose of the composition comprising a compound of Formula I and a pharmaceutically acceptable carrier and the equivalent test subject is administered a single dose of the molar equivalent composition comprising pirfenidone under the same dosing conditions. In an even more specific embodiment, the test subject is the same individual as the equivalent test subject and is simultaneously administered a single dose of a composition comprising a compound of Formula I, a molar equivalent amount of pirfenidone and a pharmaceutically acceptable carrier.

In a related embodiment, the invention provides a pharmaceutical composition comprising a compound of Formula I and pharmaceutically acceptable carrier, wherein the serum terminal elimination half-life of the compound following IV administration of the composition to a test subject is greater than 1.2 hours, greater than 1.4 hours, greater than 1.5 hours, greater than 2 hours, greater than 3 hours, or greater than 3.5 hours. In a more specific embodiment, the serum terminal elimination half-life values are determined after administration of a single dose of the composition.

In a related embodiment, the invention provides a pharmaceutical composition comprising a compound of Formula I and a pharmaceutically acceptable carrier, wherein the serum terminal elimination half-life of the compound following administration of a single dose of the pharmaceutical composition to a mammal, preferably a human, is greater than the serum terminal elimination half-life of pirfenidone when pirfenidone is administered to an equivalent test subject in a molar equivalent pharmaceutical composition under the same dosing conditions as the compound of Formula I.

In other embodiments, the serum terminal elimination half life of a compound of Formula I produced by administration of a pharmaceutical composition of this invention is greater than 2 hours, greater than 3 hours, or greater than 3.5 hours. In a more specific embodiment, the compound of Formula I is administered in a single dose.

In another embodiment, the invention provides a pharmaceutical composition comprising a compound of Formula I and a pharmaceutically acceptable carrier, the administration of which to a test subject results in an AUC_{0-∞} of the compound that is greater than the AUC_{0-∞} of pirfenidone when pirfenidone is administered to an equivalent test subject in a molar equivalent pharmaceutical composition under the same dosing conditions as the compound of Formula I. In a more specific embodiment, the test subject is administered a single dose of the composition comprising a compound of Formula I and the equivalent test subject is administered a single dose of the molar equivalent composition comprising pirfenidone under the same dosing conditions. In an even more specific embodiment, the test subject is the same individual as the equivalent test subject and is simultaneously administered a single dose of a composition comprising a compound of Formula I, a molar equivalent amount of pirfenidone and a pharmaceutically acceptable carrier.

In other embodiments, the AUC_{0-∞} produced by a pharmaceutical composition of this invention is at least 110%, 120%, 130%, 140%, 150%, 160%, 170%, or more of the AUC_{0-∞} produced by a molar equivalent pirfenidone composition administered under the same dosing conditions. In a more specific embodiment, the AUC_{0-∞} values are determined after administration of a single dose of the composition.

In another embodiment, the invention provides a pharmaceutical composition comprising a compound of Formula I and a pharmaceutically acceptable carrier, the oral administration of which to a test subject results in a maximum serum concentration of the compound (Cₘₐₓ) that is greater than the maximum serum concentration of pirfenidone when pirfenidone is orally administered to an equivalent test subject in a molar equivalent pharmaceutical composition under the same dosing conditions as the compound of Formula I. In a more specific embodiment, the test subject is administered a single dose of the oral composition comprising a compound of Formula I and the equivalent test subject is administered a single dose of the molar equivalent oral composition comprising pirfenidone under the same dosing conditions. In an even more specific embodiment, the test subject is the same individual as the equivalent test subject and is administered a single dose of an oral composition comprising a compound of Formula I, a molar equivalent amount of pirfenidone and a pharmaceutically acceptable carrier.

In a related embodiment, the maximum serum concentration a compound of Formula I produced by oral administration of a pharmaceutical composition of this invention is at least 120%, 130%, 140%, 150%, 160% or more of the maximum serum concentration of pirfenidone produced by oral administration of a molar equivalent pirfenidone composition administered under the same dosing conditions. In a more specific embodiment, the Cₘₐₓ values are determined after administration of a single oral dose of the composition.

The compounds of the present invention also demonstrate greater resistance to certain metabolism as compared to pirfenidone. Thus, in another embodiment, the invention provides a pharmaceutical composition comprising a compound of Formula I and a pharmaceutically acceptable carrier, wherein the rate of serum clearance of the compound following IV dosing is less than the rate of serum clearance of pirfenidone following intravenous administration of pirfenidone to an equivalent test subject in a molar equivalent pharmaceutical composition and under the same dosing conditions as the compound of Formula I. In other embodiments, the rate of serum clearance of a compound following IV administration of a composition of this invention is less than 90%, less than 80%, or less than 70% of the serum clearance rate of pirfenidone following IV administration of a molar equivalent pirfenidone composition to an equivalent test subject administered under the same dosing conditions. In a more specific embodiment, the test subject is administered a single dose of the IV composition comprising a compound of Formula I and the equivalent test subject is administered a single dose of the molar equivalent IV composition comprising pirfenidone. In an even more specific embodiment, the test subject is the same individual as the equivalent test subject and is simultaneously administered a single dose of an IV composition comprising a compound of Formula I, a molar equivalent amount of pirfenidone and a pharmaceutically acceptable carrier.

In a related embodiment, the invention provides a pharmaceutical composition comprising a compound of Formula I and a pharmaceutically acceptable carrier, wherein the rate of serum clearance of the compound following IV administration of a single dose of the composition to a test subject is about 200 to about 375, about 225 to about 350, or about 250 to about 325 ml/h/kg. In a more specific embodiment, the test subject is a chimpanzee.

In yet another embodiment, the invention provides a pharmaceutical composition comprising a compound of Formula I and a pharmaceutically acceptable carrier, the administration of which to a test subject results in at least one of: a) a similar steady state AUC_{0-∞}; b) a similar steady state Cₘₐₓ; or c) a similar steady state Cₘᵢₙ (minimum serum concentration of a compound) as compared to pirfenidone when pirfenidone is administered to an equivalent test subject in a pharmaceutical composition comprising an amount of pirfenidone that is greater than the amount of the compound of Formula I on a mole basis of active ingredient and that is administered under the same dosing conditions as the compound of Formula I. In a more specific embodiment, the test subject is administered a single dose of the IV composition comprising a compound of Formula I and the equivalent test subject is administered a single dose of the molar equivalent IV composition comprising pirfenidone under the same dosing conditions. In an even more specific embodiment, the test subject is the same individual as the equivalent test subject and is simultaneously administered a single dose of an IV composition comprising a compound of Formula I, a molar equivalent amount of pirfenidone and a pharmaceutically acceptable carrier.

In other embodiments, the effective amount of a compound of Formula I required per day is no more than 80%, 70%, 60%, 50%, 40%, or less of the amount of pirfenidone on a mole basis of active ingredient required per day to produce a similar steady state AUC_{0-∞}, a similar steady state Cₘₐₓ and/or a similar steady state Cₘᵢₙ when administered under the same dosing conditions as the compound of Formula I. In a more specific embodiment, the compound of Formula I is administered once daily.

In a more specific embodiment, in each of the compositions set forth above, the compound is selected from Compound 106 and Compound 108.

The term “molar equivalent amount” as used herein means an amount present in a first composition that is the same as the amount present in a second composition on a mole basis of active ingredient.

A “test subject” is any mammal, preferably a chimpanzee or a human.

An “equivalent test subject” is defined herein as being of the same species and sex as the test subject, in the same fed/fasting state as the test subject and which shows no more than 10% variability as compared to the test subject in the pharmacokinetic parameter being tested after administration of an equal amount of pirfenidone to both the test subject and the equivalent subject. In certain embodiments, an “equivalent test subject” is the same individual as the “test subject.”

As used herein, “under the same dosing conditions” means that the pharmaceutical compositions being compared contain the same carriers and excipients and are administered using the same route and frequency.

As used herein, “similar steady state AUC_{0-∞}” means that the steady state AUC_{0-∞} values being compared are within 5% of each other. For example, within 3%, such as within 2%.

As used herein, “similar steady state Cₘₐₓ” means that the steady state Cₘₐₓ values being compared are within 5% of each other. For example, within 3%, such as within 2%.

As used herein, “similar steady state Cₘᵢₙ” means that the steady state Cₘᵢₙ values being compared are within 5% of each other. For example, within 3%, such as within 2%.

### Methods of Treatment

In another embodiment, the invention provides a method of inhibiting the production and activity of TNF-alpha and TGF-beta in a cell, comprising contacting a cell with one or more compounds of Formula I herein.

According to another embodiment, the invention provides a method of treating a disease that is beneficially treated by pirfenidone in a patient in need thereof comprising the step of administering to said patient an effective amount of a compound or a composition of this invention. Such diseases are well known in the art and are disclosed in, but not limited to the following patents and published applications: WO 2001058448, WO 2003051388, WO 2004019863, WO 2004073713, WO 2004105684, WO 2005039598, WO 2005038056, WO 2005110478, and WO 2007053610.

Such diseases include, but are not limited to, idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; glomerulosclerosis (FSGS); radiation-induced fibrosis; multiple sclerosis, including secondary progressive multiple sclerosis; uterine leiomyomas (fibroids); alcoholic liver disease including hepatic steatosis, hepatic fibrosis and hepatic cirrhosis; keloid scarring; hepatitis C virus (HCV) infection; proliferative disorders, including angiogenesis-mediated disorders, cancer (including glioma, glioblastoma, breast cancer, colon cancer, melanoma and pancreatic cancer) and fibrotic disorders; interstitial lung diseases; atrial fibrillation (AF); organ transplant rejection; and scleroderma and related fibrotic conditions of the skin.

In one particular embodiment, the method of this invention is used to treat a disease or condition selected from idiopathic pulmonary fibrosis, neurofibromatosis, Hermansky-Pudlak syndrome, diabetic nephropathy, renal failure, hypertrophic cardiomyopathy (HCM), glomerulosclerosis (FSGS), radiation-induced fibrosis, multiple sclerosis, and uterine leiomyomas (fibroids) in a patient in need thereof.

In another particular embodiment, the method of the invention is used to treat renal fibrosis, hepatic fibrosis, uterine leiomyomas, keloid scarring, multiple sclerosis, radiation-associated fibrosis, organ transplant rejection, or cancer in a patient in need thereof.

In still another particular embodiment, the method of this invention is used to treat idiopathic pulmonary fibrosis in a patient in need thereof. In one aspect of this embodiment, the amount of the compound of this invention administered to the patient is from about 900 to about 1750 mg/day.

In another particular embodiment, the method of this invention is used to treat secondary progressive multiple sclerosis in a patient in need thereof. In one aspect of this embodiment, the amount of the compound of this invention administered to the patient is in the range of from about 900 to about 2350 mg/day.

In another particular embodiment, the method of this invention is used to treat pancreatic cancer in a patient in need thereof.

In another more particular embodiment, the method of this invention is used to treat renal fibrosis in a patient in need thereof. More particularly the method is used to treat renal fibrosis as the result of diabetic nephropathy, glomerulopathy/FSGS or hypertension-related nephropathy. In one aspect of this embodiment, the amount of the compound of this invention administered to the patient is from about 900 to about 2350 mg/day.

In another embodiment, the amount of the compound of this invention administered to treat radiation fibrosis in a patient in need thereof is from about 900 to about 2350 mg/day.

In still another embodiment, the amount of the compound of this invention administered to treat hepatic fibrosis in a patient in need thereof is in the range of from 600 to about 1150 mg/day.

Methods delineated herein also include those wherein the patient is identified as in need of a particular stated treatment. Identifying a patient in need of such treatment can be in the judgment of a patient or a health care professional and can be subjective (e.g. opinion) or objective (e.g. measurable by a test or diagnostic method).

In another embodiment, any of the above methods of treatment comprises the further step of co-administering to said patient one or more second therapeutic agents. The choice of second therapeutic agent may be made from any second therapeutic agent known to be useful for co-administration with pirfenidone. The choice of second therapeutic agent is also dependent upon the particular disease or condition to be treated. Examples of second therapeutic agents that may be employed in the methods of this invention are those set forth above for use in combination compositions comprising a compound of this invention and a second therapeutic agent.

The term “co-administered” as used herein means that the second therapeutic agent may be administered together with a compound of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound of this invention. In such combination therapy treatment, both the compounds of this invention and the second therapeutic agent(s) are administered by conventional methods. The administration of a composition of this invention, comprising both a compound of the invention and a second therapeutic agent, to a patient does not preclude the separate administration of that same therapeutic agent, any other second therapeutic agent or any compound of this invention to said patient at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment of the invention, where a second therapeutic agent is administered to a subject, the effective amount of the compound of this invention is less than its effective amount would be where the second therapeutic agent is not administered. In another embodiment, the effective amount of the second therapeutic agent is less than its effective amount would be where the compound of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a compound of Formula I alone or together with one or more of the above-described second therapeutic agents in the manufacture of a medicament, either as a single composition or as separate dosage forms, for treatment or prevention in a patient of a disease, disorder or symptom set forth above. Another aspect of the invention is a compound of Formula I for use in the treatment or prevention in a patient of a disease, disorder or symptom thereof delineated herein.

### Diagnostic Methods and Kits

The compounds and compositions of this invention are also useful as reagents in methods for determining the concentration of pirfenidone in solution or biological sample such as plasma, examining the metabolism of pirfenidone and other analytical studies.

According to one embodiment, the invention provides a method of determining the concentration, in a solution or a biological sample, of pirfenidone, comprising the steps of:
a) adding a known concentration of a compound of Formula I to the solution of biological sample;
b) subjecting the solution or biological sample to a measuring device that distinguishes pirfenidone from a compound of Formula I;
c) calibrating the measuring device to correlate the detected quantity of the compound of Formula I with the known concentration of the compound of Formula I added to the biological sample or solution; and
d) measuring the quantity of pirfenidone in the biological sample with said calibrated measuring device; and
e) determining the concentration of pirfenidone in the solution of sample using the correlation between detected quantity and concentration obtained for a compound of Formula I.

Measuring devices that can distinguish pirfenidone from the corresponding compound of Formula I include any measuring device that can distinguish between two compounds that differ from one another only in isotopic abundance. Exemplary measuring devices include a mass spectrometer, NMR spectrometer, or IR spectrometer.

In another embodiment, the invention provides a method of evaluating the metabolic stability of a compound of Formula I comprising the steps of contacting the compound of Formula I with a metabolizing enzyme source for a period of time and comparing the amount of the compound of Formula I with the metabolic products of the compound of Formula I after the period of time.

In a related embodiment, the invention provides a method of evaluating the metabolic stability of a compound of Formula I in a patient following administration of the compound of Formula I. This method comprises the steps of obtaining a serum, urine or feces sample from the patient at a period of time following the administration of the compound of Formula I to the subject; and comparing the amount of the compound of Formula I with the metabolic products of the compound of Formula I in the serum, urine or feces sample.

The present invention also provides kits for use to treat idiopathic pulmonary fibrosis, neurofibromatosis, Hermansky-Pudlak syndrome, diabetic nephropathy, renal fibrosis, hepatic fibrosis, keloid scarring, hypertrophic cardiomyopathy (HCM), glomerulosclerosis (FSGS), radiation-induced fibrosis, multiple sclerosis, organ rejection, cancer, and uterine leiomyomas (fibroids). These kits comprise (a) a pharmaceutical composition comprising a compound of Formula I or a salt, hydrate, or solvate thereof, wherein said pharmaceutical composition is in a container; and (b) instructions describing a method of using the pharmaceutical composition to treat one or more of the aforementioned disease or conditions.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example a conventional cardboard box would not generally be used to hold a liquid suspension. It is feasible that more than one container can be used together in a single package to market a single dosage form. For example, tablets may be contained in a bottle, which is in turn contained within a box. In one embodiment, the container is a blister pack.

The kits of this invention may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the kits of this invention may comprise in a separate vessel of container a pharmaceutical composition comprising a second therapeutic agent, such as one of those listed above for use for co-administration with a compound of this invention.

The invention now being generally described, it will be more readily understood by reference to the following examples which are included merely for purposes of illustration of certain aspects and embodiments of the present invention, and are not intended to limit the invention in any way.

### EXAMPLES

### Example 1

### 6-Deutero-5-methyl-1-phenylpyridin-2(1H)-one (Formula I, R³ is D)

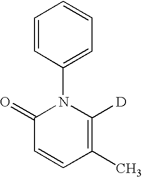

To a reaction vessel was added pirfenidone (25 mg, 0.135 mmol), CD₃OD (Cambridge Isotopes, 99.8 atom % D, 1 mL), 40% w/w NaOD in D₂O (Aldrich, 99.5 atom % D, 0.5 mL), and a stirbar. The reaction mixture was heated with vigorous stirring at 65° C. for 16 h. The reaction mixture was quenched via dropwise addition of 35% w/w DCl in D₂O (Aldrich, 99 atom % D, 0.75 mL). The resulting milky opaque mixture was diluted with water and the resulting clear colorless solution was transferred to a separatory funnel. The solution was extracted twice with CHCl₃ (10 mL, 5 mL). The organic layers were combined, dried over magnesium sulfate, filtered and concentrated on a rotary evaporator to yield the title compound as a clear colorless residue (28 mg). ¹H NMR (300 MHz, CDCl₃) δ 2.10 (s, 3H), 6.67 (d, J=9.3, 1H), 7.28 (d, J=9.3, 1H), 7.36-7.49 (m, 5H). LCMS m/z 187.1 [M+H]. ¹H NMR integration indicated the presence of hydrogen at the R³ position as 29% relative to the protio compound.

### Example 2

### 3-Deutero-5-methyl-1-phenylpyridin-2(1H)-one (Formula I, R¹ is D)

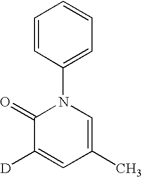

To a microwave reactor vial was added pirfenidone (20 mg, 0.108 mmol), 35% w/w DCl in D₂O (Aldrich, 99 atom % D, 1.25 mL), and a stirbar. The vial was sealed and the clear colorless solution was heated in a Biotage Personal Chemistry microwave reactor for 30 min at 170° C. The vial was cooled to room temperature (rt) and the reaction mixture was transferred to a separatory funnel. The mixture was diluted with water and CH₂Cl₂. The acidic aqueous layer was neutralized via the careful addition of 5N aqueous NaOH. The layers were shaken and separated. The aqueous layer was extracted with CH₂Cl₂ and the organic layers were combined, washed with brine, dried over magnesium sulfate, filtered and concentrated on a rotary evaporator to yield the title compound as a clear colorless residue. ¹H NMR (300 MHz, CDCl₃) δ 2.11 (s, 3H), 7.12 (m, 1H), 7.28 (m, 1H), 7.36-7.50 (m, 5H). LCMS m/z 186.9 [M+H]. ¹H NMR integration indicated the presence of hydrogen at the R¹ position as 0.1% relative to the protio compound.

### Example 3

### 3,6-Dideutero-5-(methyl-d₃)-1-phenylpyridin-2(1H)-one (Compound 106)

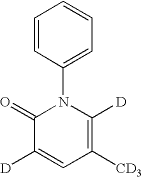

To a thick-walled glass pressure vessel flushed with nitrogen was added pirfenidone (100 mg, 0.540 mmol), D₂O (Cambridge Isotopes, 99.9 atom % D, 1.2 mL), and a stir bar. To the stirring slurry was then added 35% w/w DCl in D₂O (Aldrich, 99 atom % D, 0.135 mL, 1.64 mmol) and the solids began to partially dissolve. To the mixture was added 10% palladium on carbon (10 mg, 110% w/w of pirfenidone) and the vessel was flushed once more with nitrogen. The vessel was then flushed with hydrogen and sealed. The vessel was heated in a 160° C. oil bath for 16.5 hours with stirring (this reaction time varies with reaction scale). The vessel was cooled to rt and flushed with nitrogen. The reaction mixture was diluted with CH₂Cl₂ (25 mL), stirred vigorously, and filtered through a 0.45 micron syringe filter. The palladium residue in the filter was flushed with CH₂Cl₂ (50 mL) and the combined filtrate bilayer was poured into a separatory funnel. Saturated aqueous sodium bicarbonate (50 mL) was added and the layers were shaken and separated. The organic layer was washed with brine, dried over magnesium sulfate, filtered and concentrated on a rotary evaporator to afford the title compound as a clear, colorless residue (57 mg). The residue solidified upon standing. ¹H NMR (300 MHz, CDCl₃) δ 7.26 (s, 1H), 7.36-7.50 (m, 5H). ¹H NMR (300 MHz, THF-d₈) δ 7.20 (s, 1H), 7.30-7.43 (m, 5H). LCMS m/z 191.0 [M+H]. This method produced batches of Compound 106 for which ¹H NMR integration indicated the presence of hydrogen at the methyl group as 15%-26% relative to the protio compound; at the R¹ position as 2%-7% relative to the protio compound; and at the R³ position as 5%-12% relative to the protio compound.

### Example 4

### 5-(Methyl-d₃)-1-phenylpyridin-2(1H)-one (Compound 108)

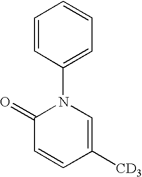

Compound 108 was synthesized according to Scheme 5, above. Details of the synthesis are set forth below.

Step 1. 5-(Methyl-d₃)-pyridin-2(1H)-one (18). To a round-bottom flask was added commercially-available 6-oxo-1,6-dihydropyridine-3-carbonitrile (17, 1.00 g, 8.33 mmol), sodium dodecylsulfate (“SDS”, 240 mg, 0.833 mmol), and 10% palladium on carbon (300 mg). Water (20.8 mL), n-butanol (20.8 mL), and 10% aqueous H₂SO₄ (4.43 mL, 8.33 mmol) were added with stirring. The vessel was flushed first with nitrogen, then with deuterium gas (Aldrich, 99.9 atom % D). The reaction was stirred at rt under a balloon of deuterium gas for 2-3 days. The vessel was flushed with nitrogen, the slurry was filtered and the palladium residue was washed well with n-butanol. The filtrate was transferred to a separatory funnel and the layers were shaken and separated. The aqueous layer was adjusted to approximately pH=5 via careful addition of 1N aqueous NaOH. The organic and aqueous layers were recombined in the separatory funnel, shaken, and separated. The aqueous layer was extracted with n-butanol (2×25 mL) and the combined organic layers were concentrated on a rotary evaporator to a minimum volume of residue. The material was diluted with 5% methanol in dichloromethane, filtered through a short silica plug and eluted with 5% methanol in dichloromethane. The product fractions were purified via column chromatography on an ISCO instrument (0% to 5% methanol in dichloromethane) to afford 303 mg of 18. ¹H NMR (300 MHz, CDCl₃) δ 6.53 (d, J=9.3, 1H), 7.14 (d, J=2.5, 1H), 7.33 (dd, J=2.3, 9.1, 1H), 13.10 (br s, 1H). LCMS m/z 113.2 [M+H].

Step 2. 5-(Methyl-d₃)-1-phenylpyridin-2(1H)-one (Compound 108). In a pressure vessel, 18 (300 mg, 2.68 mmol) was stirred in dioxane (4 mL) at rt. Iodobenzene (359 uL, 3.22 mmol) and copper (I) iodide (102 mg, 0.536 mmol) were added and the resulting slurry was stirred for 5-10 min to finely disperse the solids. N,N′-dimethylethylenediamine (115 uL, 1.07 mmol) and K₃PO₄ (1.14 g, 5.36 mmol) were added and the vessel was flushed with nitrogen and sealed. The blue slurry was heated overnight in a 110° C. oil bath. The resulting ochre slurry was cooled, diluted with water (15 mL), and transferred to a separatory funnel. The mixture was extracted with EtOAc (3×50 mL). The combined organic layers were washed with brine, dried over magnesium sulfate, filtered and concentrated on a rotary evaporator to afford a pale green oil. Purification via column chromatography on an ISCO instrument (0% to 3% methanol in dichloromethane) provided 427 mg of Compound 108. ¹H NMR (300 MHz, CDCl₃) δ 6.61 (d, J=9.3, 1H), 7.11 (d, J=2.5, 1H), 7.26 (dd, J=2.5, 9.3, 1H), 7.35-7.50 (m, 5H). LCMS m/z 189.1 [M+H]. ¹H NMR integration indicated the presence of hydrogen at the methyl group as 10% relative to the protio compound.

### Example 5

### 5-(Methyl-d₃)-1-(phenyl-d₅)-pyridin-2(1H)-one (Compound 109)

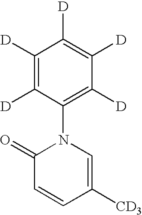

To a round-bottom flask were added 5-(methyl-d₃)-pyridin-2(1H)-one (18, 0.522 g, 4.65 mmol, prepared as shown in Example 6, below), K₃PO₄ (1.98 g, 9.31 mmol), and CuI (177 mg, 0.931 mmol) under N₂. Toluene (7.8 mL) was added with stirring. Iodobenzene-d₅ (CDN Isotopes, 99.7 atom % D, 0.613 mL, 5.59 mmol) was added, followed by N,N-dimethylethylenediamine (0.201 mL, 1.86 mmol). The heterogeneous reaction mixture was heated to reflux for 3 h. The mixture was then cooled to 75° C. and filtered through a pad of Celite. The filter cake was washed twice with hot (75° C.) toluene. The filtrate was transferred to a separatory funnel, and washed with water (3×). The combined aqueous layers were extracted with toluene (2×). The combined organic layers were washed with water (1×), aq. 1N HCl (1×) and water (1×). The combined organic layers were dried (Na₂SO₄) and concentrated to dryness. To the resulting yellow solid was added heptane (15 mL) and the mixture was stirred at room temperature for 48 h. The mixture was filtered and the filtrate dried under vacuum to afford 414 mg (46%) of Compound 109 as a white solid. ¹H NMR (300 MHz, DMSO-d₆) δ 7.48 (dd, J=0.6, 2.6, 1H), 7.43 (dd, J=2.6, 9.4, 1H), 6.46 (dd, J=0.8, 9.3, 1H). LCMS m/z 194.2 [M+H]. A signal corresponding to the protio methyl group was not detected in the ¹H NMR. A signal corresponding to the protio phenyl group was not detected in the ¹H NMR.

### Example 6

### Alternative Synthesis of 5-(Methyl-d₃)-pyridin-2(1H)-one (18) and 5-(Methyl-d₃)-1-phenylpyridin-2(1H)-one (Compound 108)

An alternative synthesis of 5-(methyl-d₃)-pyridin-2(1H)-one (18) and 5-(methyl-d₃)-1-phenylpyridin-2(1H)-one (Compound 108) is depicted in Scheme 6 and described below.

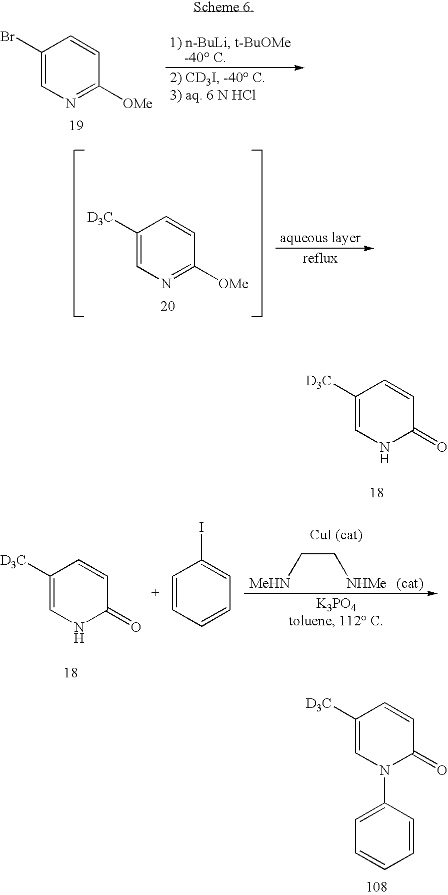

Step 1. 5-(Methyl-d₃)-pyridin-2(1H)-one (18). To a 2000 mL, 4-neck round bottom flask equipped with a mechanical stirrer, a thermocouple, and an addition funnel was added 5-bromo-2-methoxy-pyridine (19, 84.11 mL; 1 equiv), followed by t-BuOMe (1050 mL). The resulting mixture was stirred under N₂ and cooled to −39° C. using a dry-ice/acetone bath. n-BuLi was then added as a 2.5 M solution in hexane (286 mL; 1.1 equiv) via addition funnel. The addition rate was adjusted to keep the internal temperature below −30° C. The total addition time was 25 min. The resulting orange slurry was stirred for 80 min while maintaining the reaction temperature between −40° C. and −30° C. A solution of iodomethane-d₃ (46.5 mL; 1.15 equiv; Isotech, 99.5+atom % D) in t-BuOMe (126 mL) was then added via syringe at −39° C. The addition rate was adjusted to keep the internal temperature below −28° C. The total addition time was 60 min. The resulting slurry was stirred for 80 min while maintaining the reaction temperature between −40° C. and −30° C. The cold bath was then removed, and the reaction mixture was allowed to warm to 15° C. over a period of 65 min. This produced an orange slurry comprising 5-(methyl-d₃)-2-methoxypyridine 20, which was not purified prior to the next step as described below.

The orange slurry containing 5-(methyl-d₃)-2-methoxypyridine 20 was filtered through a pad of Celite pre-wetted with t-BuOMe. The flask was rinsed with t-BuOMe (2×125 mL). The rinses were used to further wash the Celite cake. The yellow filtrate was transferred to a 2 L separatory funnel and then was washed with aqueous 6 N HCl (3×475 mL and 2×250 mL). The combined aqueous layers were then washed with heptane (3×250 mL). The aqueous layer was transferred to a 2 L, 3-neck round bottom flask equipped with a magnetic stirrer, thermocouple and a condenser. The aqueous layer was heated to reflux (109° C.) for 25 h, and then was allowed to cool to rt overnight. An aliquot was sampled and analyzed by HPLC to show clean transformation with a product conversion rate of 98.7% by HPLC. The aqueous layer was then cooled to 5° C. in an ice bath and was neutralized with 50 w/w % aq. NaOH while keeping the internal temperature below 30° C. The pH change was monitored using a pH meter. Neutralization was completed when the pH was 7.02. The total amount of 50 w/w % aq. NaOH used was less than approximately 390 g.

The neutralized solution was then filtered through a pad of water-wetted Celite to remove a minor amount of a dark brown solid. The filtrate which was obtained was yellow. Vacuum suction was used to dry the wet Celite cake. The pH of the filtrate was readjusted to 7.02 using aqueous 1 N HCl. Most of the remaining water was removed under vacuum (50-70 mm Hg) at 70° C. The residue became saturated with NaCl when 900 mL water was removed, and the remaining water volume was about 400 mL. The aqueous solution was then decanted into a 2 L separatory funnel, while the solid residue was washed with CH₂Cl₂ (300 mL). The CH₂Cl₂ layer was also transferred to the separatory funnel. The aqueous layer that was remaining in the separatory funnel was extracted with the CH₂Cl₂. This solid wash with CH₂Cl₂ and extraction was repeated 5 more times. The combined organic layer (total volume=1.8 L) was washed with water (250 mL). The phase separation was observed to be slow. The lower organic layer was cloudy and became clear after standing overnight. The organic layer was collected and concentrated in vacuo to dryness to obtain 53.25 g of the crude product as a light yellow solid (“Crop 1”).

The aqueous layers were combined and concentrated in vacuo to dryness (50-70 mmHg, 70° C.) to obtain a solid residue. The residue was taken up in CH₂Cl₂ (300 mL) and stirred for 30 min. The mixture was filtered through a core-porosity funnel to obtain a clear filtrate, which was concentrated in vacuo to dryness to obtain 19.87 g of additional product as a yellow solid (“Crop 2”).

The two crops of product were analyzed by HPLC to show that Crop 1 had a product purity of 93.5% and Crop 2 had a product purity=99.7%. The two crops were then combined and added to a 3-neck, 1 L round bottom flask equipped with mechanical stirrer, thermocouple, and a condenser. t-BuOMe (400 mL) and CH₂Cl₂ (100 mL) were added and the resulting mixture was heated to reflux (54° C.) under N₂ for 3.5 hours, then cooled to rt while maintaining stirring over the weekend. The resulting slurry was filtered through a core-porosity funnel and the solid was washed with t-BuOMe (100 mL) and then air-dried to obtain 63.92 g of the product as a light tan solid with an HPLC purity of 98.5%.

The filtrate was then concentrated in vacuo to dryness to afford 8.35 g of additional product as a yellow residue with an HPLC purity of 51%.

The light tan product solid was transferred to a 1 L Erlenmeyer flask, and toluene (625 mL) was added. The mixture was heated to reflux to dissolve the solid. The mixture was then gradually cooled to ambient temperature and then to 10° C. in an ice bath. The mixture was then filtered through a core-porosity funnel. The solid was washed with t-BuOMe (2×50 mL) and air-dried under vacuum to afford 57.07 g of the product as a light tan solid. Analysis: ¹H NMR (400 MHz) showed no aliphatic peaks corresponding to 5-CHₓD₃₋ₓ-2-pyridone (where x=1, 2, or 3). Deuterium incorporation >99%, as defined by deuterium purity of CD₃I. Chemical purity by HPLC=99.1%.

Step 2. 5-(Methyl-d₃)-1-phenylpyridin-2(1H)-one (Compound 108). To a 2 L, 3-necked round-bottom Morton flask equipped with a mechanical stirrer, a heating mantle, and a condenser was added 5-(methyl-d₃)-pyridin-2(1H)-one (18, 55 g, 490 mmol, 1 equiv, >99% pure), K₃PO₄ (208 g, 980.92 mmol, 2 equiv), and CuI (18.68 g, 98.09 mmol, 0.2 equiv) under nitrogen gas. Toluene (825 mL) was then added, and the resulting mixture was agitated as iodobenzene (65.61 g, 588.55 mmol, 1.2 equiv) was added via syringe. N,N′-dimethylethylenediamine (21.14 g, 196.18 mmol, 0.4 equiv) was then added via another syringe. The resulting heterogeneous reaction mixture was then heated to reflux (112° C.) under nitrogen. After 3 h, HPLC analysis showed a conversion rate to the desired product of 98.8%.

The reaction mixture was removed from the heating mantle, cooled to 75° C., and then filtered through a pad of Celite pre-wetted with toluene. The collected wet cake was washed with hot toluene (75° C., 2×125 mL). Residual liquid was removed from the wet cake with vacuum suction. A blue residue remained on the filter cake. The residue and the filter cake were retained.

The filtrate was then transferred to a 2 L separatory funnel and washed with water (3×250 mL). The combined aqueous layers were extracted with toluene (2×150 mL). The remaining aqueous layer was dark blue in color and was retained for further extraction. The combined toluene layers were then washed with water (300 mL), aqueous 1 N HCl (300 mL) and water (300 mL). The first water wash showed a light blue color in the aqueous layer. The diluted acid wash showed a light brown color in the aqueous layer. The last water wash was nearly colorless in the aqueous layer. These two light blue and colorless water layers were retained for further extraction. The toluene layer was concentrated in vacuo to near dryness, leaving a light yellow solid. Heptane (500 mL) was added to this solid, and the resulting mixture was stirred at rt under N₂ overnight. The mixture was filtered, then air dried under vacuum to afford 60.3 g of a white solid (“First Batch”) which was found to be 99.4% pure by HPLC.

The retained dark blue aqueous layer was then back extracted with CH₂Cl₂ (2×125 mL) to obtain additional crude product. Although both liquid phases were very dark in color, there was a discernable separation line between them. The retained light blue and colorless water layers were also extracted with CH₂Cl₂ (125 mL for each). The combined CH₂Cl₂ layers were washed with water (3×150 mL), aqueous 1N HCl (200 mL) and water (150 mL). A greenish color present in the CH₂Cl₂ layer disappeared after these water washes. The CH₂Cl₂ layer was then concentrated in vacuo to afford 11.91 g of a light brown oil that solidified quickly upon standing (“Second Batch”).

Residual product was then collected from the blue residue on the wet filter cake. The blue residue was transferred back to the reaction flask. Water (700 mL) and toluene (500 mL) were added. The resulting mixture was stirred mechanically for 40 min, and then was filtered through the same Celite cake. Both the flask and the Celite cake were washed with toluene (2×100 mL). The resulting dark-blue toluene filtrate was transferred to the separatory funnel and was washed with water (300 mL), aqueous 1 N HCl (300 mL) and water (300 mL). The toluene was removed in vacuo to afford 4.14 g of a yellow solid (“Third Batch”).

The combined second and third batches of solid were then transferred to a 1 L round bottom flask and heptane (300 mL) was added. The resulting mixture was stirred vigorously at rt under N₂ overnight and then was filtered to provide 15.2 g of the product (“Fourth Batch”) as a cream-white solid with an HPLC purity profile of 99.1%.

The first and fourth batches of solid were combined, taken up in heptane (350 mL), and then filtered. The solid was dried under vacuum to provide the product (Compound 108) as a white solid, weight (74.31 g, 80.5% yield) with a purity of 99.65% by HPLC. A signal corresponding to the protio methyl group was not detected in the ¹H NMR (400 MHz).

### Example 7

### Synthesis and Isolation of 5-(methyl-d₃)-2-methoxypyridine (20)

In order to obtain isolated 5-(methyl-d₃)-2-methoxypyridine 20, the first step of Scheme 6 was modified as follows.

To a 100 mL round-bottom flask equipped with a magnetic stirrer and thermocouple was added 5-bromo-2-methoxy-pyridine (5.83 g, 31 mmol, 1 eq) and t-BuOMe (50 mL). The solution was cooled to −40° C. An n-BuLi solution (21.31 mL, 1.6 M in hexane, 34.1 mmol, 1.1 eq) was then added via a syringe. The addition rate was adjusted to keep the internal temperature below −30° C. The solution was added over a period of 25 min. After the addition finished, the mixture was stirred at between −40° C. and −35° C. for 90 min. The resulting light pink slurry in t-BuOMe (5 mL) was passed through a small pad of dry K₂CO₃. To the pinkish reaction mixture was then added iodomethane-d₃ (Isotech, 99.5+ atom % D) (5.17 g, 35.65 mmol, 1.15 eq.) via a syringe, at an addition rate to keep the internal temperature below −30° C. Addition time was 25 min. After the addition, the slurry was stirred at −30° C. for 30 min, then was warmed to 0° C., followed by addition of water (40 mL) and additional stirring for 25 min. The solution was then transferred to a separatory funnel and the aqueous layer was discarded. The organic layer was washed with 1N HCl (50 mL), and the organic layer was discarded. The remaining acidic aqueous layer was washed with t-BuOMe (2×25 mL), then with 1N NaOH (55 mL), and was then extracted into t-BuOMe (3×30 mL). The resulting organic layer was collected and washed with water (2×30 mL). The resulting organic layer was concentrated in vacuo to yield 3.0 g (77%) of 5-(methyl-d₃)-2-methoxypyridine 20 as a light yellow liquid. ¹H NMR (400 MHz, CDCl₃): 7.96 (1H, d, J=2.38 Hz), 7.38 (1H, dd, J=2.42 Hz, 8.38 Hz), 6.66 (1H, d=8.41 Hz), 3.90 (s, 3H). A signal corresponding to the protio methyl group was not detected in the ¹H NMR.

### Example 8

### Evaluation of Metabolic Stability of Compound 106 in Human Liver Microsomes

Certain in vitro liver metabolism studies have been described previously in the following references: Obach, R S, Drug Metab Disp, 1999, 27:1350; Houston, J B et al., Drug Metab Rev, 1997, 29:891; Houston, J B, Biochem Pharmacol, 1994, 47:1469; Iwatsubo, T et al, Pharmacol Ther, 1997, 73:147; and Lave, T et al., Pharm Res, 1997, 14:152.

The objectives of this study were to determine the metabolic stability of Compound 106 as compared to pirfenidone in pooled human liver microsomal incubations. Samples of the test compounds, exposed to pooled human liver microsomes, were analyzed using LC-MS/MS detection with multiple reaction monitoring (MRM) to measure the disappearance of the test compounds.

Human liver microsomes were obtained from XenoTech, LLC (Lenexa, Kans.). The incubation mixtures were prepared according to Table 2:

TABLE 2
Reaction Mixture Composition for
Human Liver Microsome Study

| | | |
|---|---|---|
| Liver Microsomes | 3.0 | mg/mL |
| Potassium Phosphate, pH 7.4 | 100 | mM |
| Magnesium Chloride | 10 | mM |

The reaction mixture of Table 2 was prepared. Two aliquots of this reaction mixture were used for test compound 106. The aliquots were incubated in a shaking water bath at 37° C. for 3 minutes. Test compound 106 was then added into each aliquot at a final concentration of 0.5 μM. The reaction was initiated by the addition of cofactor (NADPH) into one aliquot (the other aliquot (no NADPH) serving as the negative control). Both aliquots were then incubated in a shaking water bath at 37° C. Fifty microliters (50 μL) of the incubation mixtures were withdrawn in triplicate from each aliquot at multiple time points and combined with 50 μL of ice-cold acetonitrile to terminate the reaction. The same procedure was followed for pirfenidone and the positive control, 7-ethoxy coumarin. Testing was done in triplicate.

All samples were analyzed using LC-MS/MS. Surprisingly, both pirfenidone and Compound 106 were very stable in human liver microsomes. After 30 minutes of exposure, greater than 75% of each compound remained unmetabolized.

### Example 9

### Pharmacokinetics of Compound 106 After Intravenous and Oral Dosing in Rats

Male Sprague-Dawley rats (3 for each route of administration) were administered a combination of 8 mg/kg of Compound 106 and 8 mg/kg of pirfenidone in 10% DMI (dimethyl isosorbide), 15% ethanol, 35% PG in distilled water by either oral or intravenous dosing. Blood samples from the dosed rats were collected prior to dosing and at 15, 30, 45, 60, 75, 90, 120, 240, and 360 minutes post-dosing. Plasma was isolated and prepared for analysis by mixing 0.1 ml of plasma in an Eppendorf tube with 20 μL methanol and 500 μL of quetiapine (50 ng/ml as an internal standard), vortexing for 1 minute and then centrifuging at 15,000 rpm for 5 minutes to remove any cellular debris. Plasma samples were analyzed by LC-MS/MS.

LC was performed using an Agilent (Agilent Technologies Inc. USA) liquid chromatograph equipped with an isocratic pump (1100 series), an autosampler (1100 series) and a degasser (1100 series). Plasma samples (2 μL) were run at 25° C. on a Phenomenex Gemini, C18, 5 μm, (50 mm×2.0 mm) column using 0.1% formic acid:methanol (30:70) as the mobile phase with an elution rate of 300 μL/min.

Mass spectrometric analysis was performed on plasma samples (2 μL) prepared as set forth above using an API3000 (triple-quadrupole) instrument from AB Inc (Canada) with an ESI interface. The data acquisition and control system were created using Analyst 1.4 software from ABI Inc.

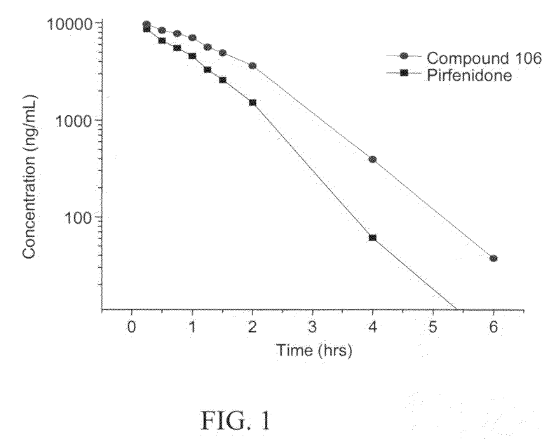

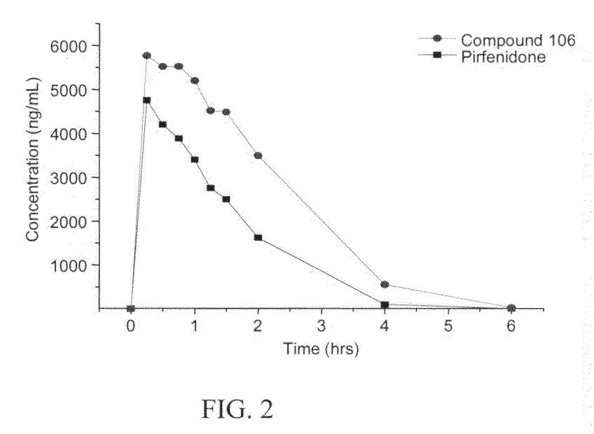

The results of the above assay are shown in FIG. 1 and FIG. 2 and are summarized in Table 3, below.

TABLE 3
Pharmacokinetics of Compound 106 in Rats

| | | | Oral Dosing |
|---|---|---|---|
| | IV Dosing | | AUC_{0-∞} |
| Compound | Half-life (hrs) | AUC_{0-∞} (ng-hrs/ml) | (ng-hrs/ml) |
| Compound 106 | 1.2 | 20056 | 13830 |
| Pirfenidone | 0.74 | 11649 | 7909 |

Compound 106 showed a 62% increase in half-life and a 72% increase in AUC as compared to pirfenidone following intravenous administration in rats. A similar effect was observed after oral dosing where Compound 106 showed a 75% increase in AUC as compared to pirfenidone.

### Example 10

### Pharmacokinetics of Compounds 106 and 108 After Intravenous and Oral Dosing in Chimpanzees

Chimpanzees (one male and one female for each route of administration) were administered a combination of 100 mg of Compound 106, 100 mg of Compound 108 and 100 mg of pirfenidone in 10% DMI (dimethyl isosorbide), 15% ethanol, 35% PG in distilled water (total volume 150 ml) by either oral or intravenous dosing. Intravenous dosing was performed by infusion over a 30 minute period. Blood samples from the orally dosed chimps were collected just prior to dosing and at 15, 30, 60, 90, 120, 240, 360, 480, 600, 720 and 1440 minutes post-dosing. Blood samples (4.5 ml) from the intravenously dosed chimps were collected just prior to infusion, at 15 and 29.5 minutes after the start of infusion, and then at 6, 15, 30, 45, 60, 120, 240, 360, 480, 600, 720 and 1440 minutes post-infusion. Plasma was isolated and prepared for analysis by mixing 0.1 ml of plasma with 300 μL of indiplon in (50 ng/ml in acetonitrile/water; 90/10; v/v) as an internal standard, vortexing and then centrifuging to remove any precipitated protein.

Plasma samples (10 μl) were injected to a Zorbax SB-C8 (Rapid Resolution) column (2.1×30 mm, 3.5 μm). The initial mobile phase condition was 100% A (water with 0.1% formic acid) and 0% B (acetonitrile with 0.1% formic acid) with a flow rate at 0.75 mL/min. Mobile phase B was allowed to reach 100% in 0.75 minutes and held for 1.0 minute before ramping back to 0% in another 0.1 minute. The overall run time was 3 minutes. The precursor/product ion pairs were set at m/z 186/92, m/z 191/97, m/z 189/95 and m/z 377/293 for detecting pirfenidone, Compound 106, Compound 108 and indiplon, respectively. Plasma concentration data for each compound in each animal was individually fitted to a 2-compartment model using WinNonLin version 5.2 (PK Model #9) with weighting of 1/y.

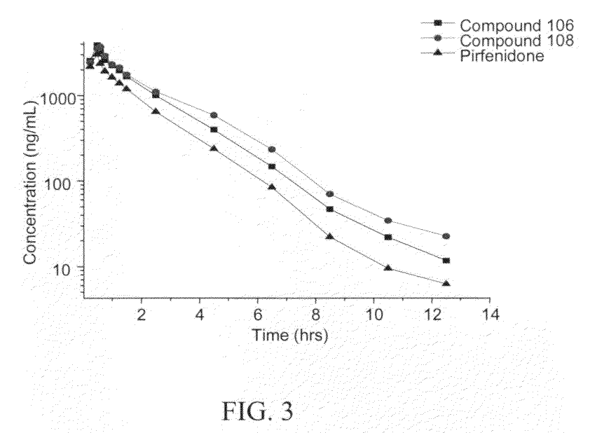

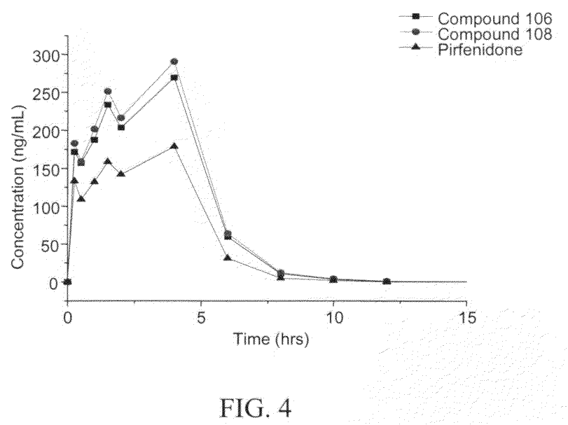

The results of the above assay are shown in FIG. 3 and FIG. 4 and are summarized in Table 4 below.

TABLE 4
Pharmacokinetics of Compound 106 and 108 in Chimps

| | IV Dosing (Avg) | | | | |
|---|---|---|---|---|---|
| | Half- | | | Oral Dosing (Avg) | |
| | life | AUC_{0-∞} | Clearance | Cₘₐₓ | AUC_{0-∞} |
| Compound | (hrs) | (ng-hrs/ml) | (ml/h/kg) | (ng/ml) | (ng-hrs/ml) |
| Compound 106 | 1.4 | 6900 | 262 | 340 | 1260 |
| Compound 108 | 1.5 | 7000 | 275 | 360 | 1350 |
| Pirfenidone | 1.2 | 4800 | 394 | 230 | 830 |

Compound 106 showed a greater than 10% increase in half-life, an almost 50% increase in AUC and a greater than 30% decrease in clearance rate as compared to pirfenidone following intravenous administration in chimps. Compound 108 also showed similar effects demonstrating a greater than 20% increase in half-life, an almost 50% increase in AUC and a greater than 30% decrease in clearance rate as compared to pirfenidone following intravenous administration in chimps.

Similar effects for the compounds of this invention were observed after oral dosing. Compound 106 showed a greater than 50% increase in AUC and Cₘₐₓ as compared to pirfenidone. Compound 108 demonstrated an almost 60% increase in AUC and Cₘₐₓ as compared to pirfenidone.

### Example 11

### Pharmacokinetics of Compound 108 and 109 After Intravenous and Oral Dosing in Rats

Male Sprague-Dawley rats (3 for each route of administration) were administered a combination of a) 8 mg/kg of Compound 109 and 8 mg/kg of pirfenidone; b) a combination of 8 mg/kg of Compound 108 and 8 mg/kg of pirfenidone; or c) a combination of 8 mg/kg of Compound 108 and 8 mg/kg of Compound 109 in 10% DMI (dimethyl isosorbide), 15% ethanol, 35% PG in distilled water by either oral or intravenous dosing. For all dosings, each compound was dissolved in 5% glucose containing 10% DMI (dimethyl isosorbide), 15% ethanol, and 35% PG in distilled water.

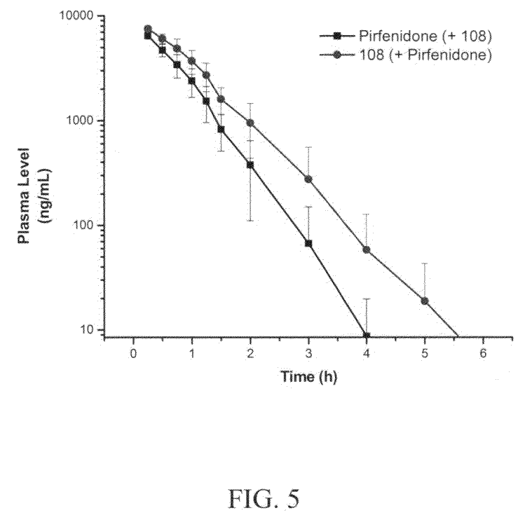

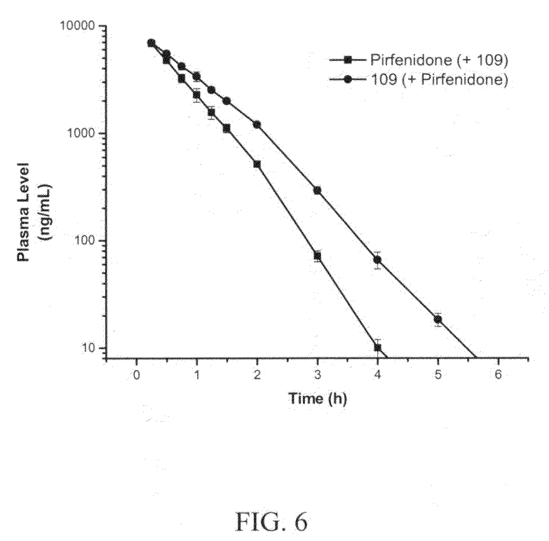

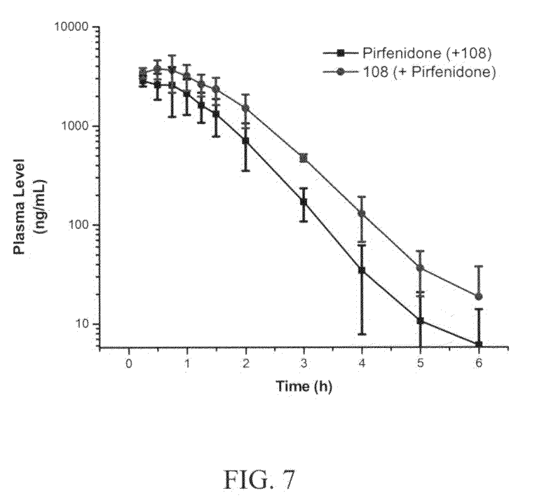

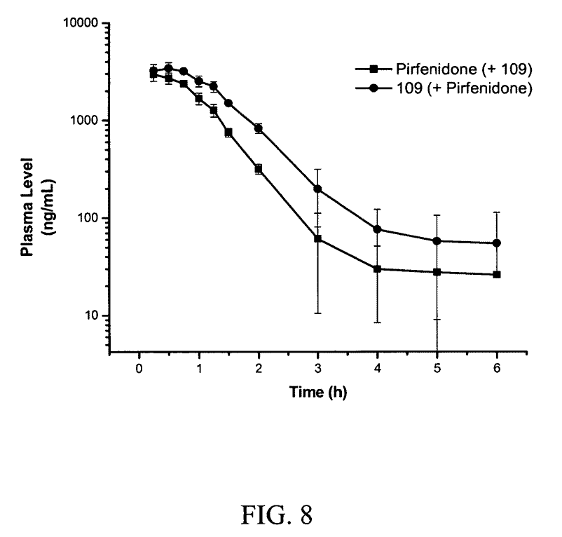

Dosing, blood sampling, plasma preparation and serum analyses were as described in Example 9. The results of these studies for combinations a) and b) above are shown in FIGS. 5 through 8, and in Table 5, below.

TABLE 5
Pharmacokinetic Comparison of Compound
108 and Compound 109 to Pirfenidone.

| % Change | | | | Oral Dosing | |
|---|---|---|---|---|---|
| compared to | IV Dosing | | | | Oral |
| Pirfenidone | Half-life | AUC_{0-∞} | Clearance | Cₘₐₓ | Bioavailability |
| Compound 108 | +37% | +42% | −29% | +31% | +15% |
| Compound 109 | +41% | +29% | −23% | +19% | +20% |

The data demonstrates that both Compound 108 and Compound 109 demonstrate a greater serum half-life and AUC_{0-∞} and a reduced rate of clearance following intravenous dosing as compared to pirfenidone. In addition both Compound 108 and Compound 109 demonstrate a greater Cₘₐₓ and oral bioavailability following oral dosing as compared to pirfenidone.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1. A compound of Formula I: 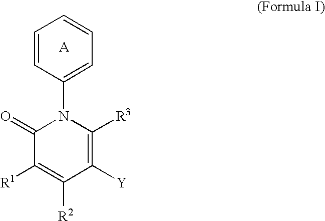
or a pharmaceutically acceptable salt thereof, wherein:
ring A is a phenyl ring having zero to five deuterium;
each of R¹, R² and R³ is independently selected from H or D;
Y is selected from CH₂D, CHD₂, and CD₃ and, when at least one of R¹, R², or R³ is D or when ring A has at least one deuterium, Y is additionally selected from CH₃.

2. The compound of claim 1, wherein Y is selected from CH₂D, CHD₂, and CD₃.

3. The compound of claim 2, wherein Y is CD₃.

4. The compound of claim 2, wherein ring A has zero or five deuterium.

5. A compound selected from: 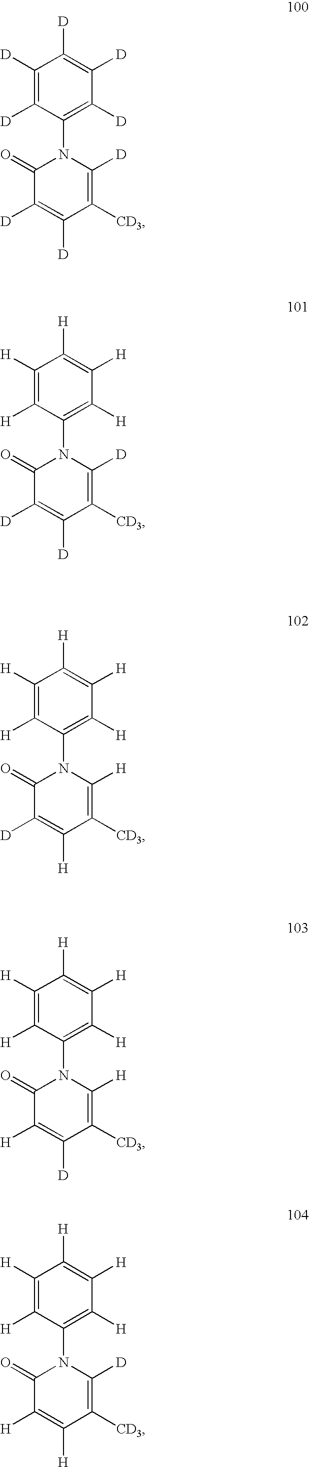 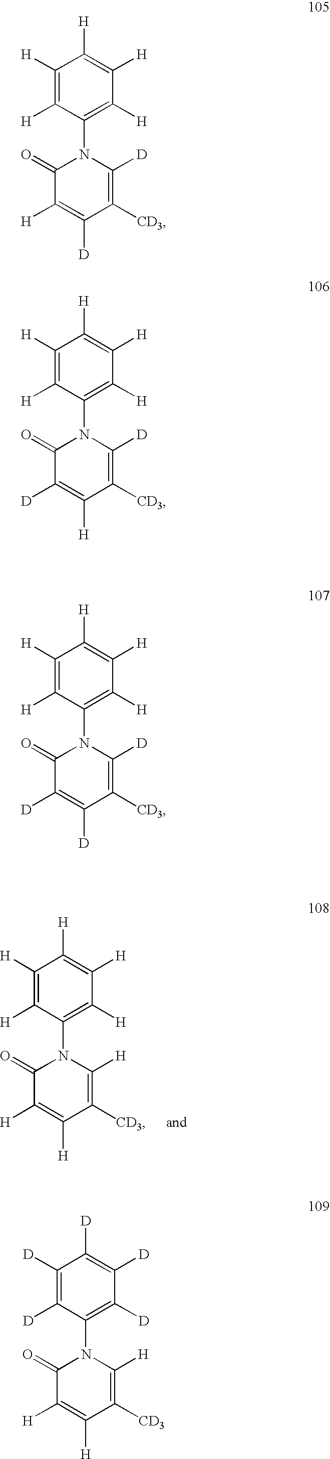
or a pharmaceutically acceptable salt of any of the foregoing.

6. The compound of any one of claims 1 to 5 or 16, wherein any atom not designated as deuterium is present at its natural isotopic abundance.

7. A pyrogen-free pharmaceutical composition comprising a compound of Formula I: 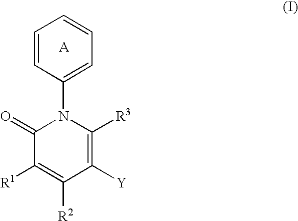
or a pharmaceutically acceptable salt thereof wherein:
ring A is a phenyl ring having zero to five deuterium;
each of R¹, R² and R³ is independently selected from H or D,
Y is selected from CH₂D, CHD₂, and CD₃ and, when at least one of R¹, R², or R³ is D or when ring A has at least one deuterium, Y is additionally selected from CH₃; and
a pharmaceutically acceptable carrier.

8. The composition of claim 7, additionally comprising a second therapeutic agent useful in the treatment of a disease or condition selected from: idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; glomerulosclerosis (FSGS); radiation-induced fibrosis; multiple sclerosis; secondary progressive multiple sclerosis; uterine leiomyomas (fibroids); alcoholic liver disease; hepatic steatosis; hepatic fibrosis; hepatic cirrhosis; keloid scarring; hepatitis C virus (HCV) infection; proliferative disorders; angiogenesis-mediated disorders; cancer;
fibrotic disorders; interstitial lung diseases; atrial fibrillation (AF); organ transplant rejection; scleroderma; and fibrotic conditions of the skin.

9. A method of inhibiting the production and/or activity of TNF-alpha and TGF-beta in a cell, comprising the step of contacting the cell with a compound of Formula I: 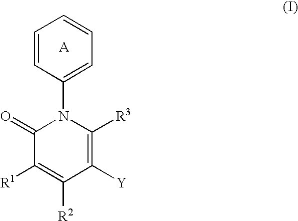
or a pharmaceutically acceptable salt thereof, wherein:
ring A is a phenyl ring having zero to five deuterium;
each of R¹, R² and R³ is independently selected from H or D;
Y is selected from CH₂D, CHD₂, and CD₃ and, when at least one of R¹, R², or R³ is D or when ring A has at least one deuterium, Y is additionally selected from CH₃.

10. A method of treating a disease selected from idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; glomerulosclerosis (FSGS); radiation-induced fibrosis; multiple sclerosis; secondary progressive multiple sclerosis; uterine leiomyomas (fibroids); alcoholic liver disease; hepatic steatosis; hepatic fibrosis; hepatic cirrhosis; keloid scarring; hepatitis C virus (HCV) infection; proliferative disorders; angiogenesis-mediated disorders; cancer;
fibrotic disorders; interstitial lung diseases; atrial fibrillation (AF); organ transplant rejection; and fibrous skin diseases, in a patient in need thereof the method comprising the step of administering to the patient a composition comprising a compound of Formula I: 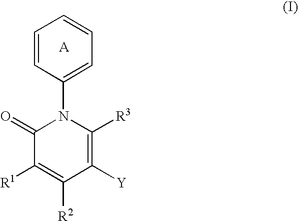
or a pharmaceutically acceptable salt thereof, wherein:
ring A is a phenyl ring having zero to five deuterium:
each of R¹, R² and R³ is independently selected from H or D;
Y is selected from CH₂D, CHD₂, and CD₃ and, when at least one of R¹, R², or R³ is D or when ring A has at least one deuterium, Y is additionally selected from CH₃ and
a pharmaceutically acceptable carrier.

11. The method of claim 10, wherein the disease or condition is selected from renal fibrosis, hepatic fibrosis, uterine leiomyomas, keloid scarring, multiple sclerosis, radiation-associated fibrosis, organ transplant rejection, and cancer.

12. The method of claim 11, wherein the disease is renal fibrosis.

13. The method of claim 12, wherein the renal fibrosis is caused by diabetic nephropathy, glomerulopathy/FSGS, or hypertension-related nephropathy.

14. The method of any one of claims 10 to 13, comprising the additional step of co-administering to the patient in need thereof a second therapeutic agent useful in the treatment of a disease or condition selected from: idiopathic pulmonary fibrosis; neurofibromatosis; Hermansky-Pudlak syndrome; diabetic nephropathy; renal fibrosis; hypertrophic cardiomyopathy (HCM); hypertension-related nephropathy; glomerulosclerosis (FSGS); radiation-induced fibrosis; multiple sclerosis; secondary progressive multiple sclerosis; uterine leiomyomas (fibroids); alcoholic liver disease; hepatic steatosis; hepatic fibrosis; hepatic cirrhosis; keloid scarring; hepatitis C virus (HCV) infection; proliferative disorders; angiogenesis-mediated disorders; cancer; fibrotic disorders; interstitial lung diseases; atrial fibrillation (AF); organ transplant rejection; scleroderma; and fibrotic conditions of the skin.

15. A compound represented by the following structure:

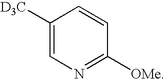

16. The compound of claim 3, wherein ring A has zero or five deuterium.
